SEQUENCE LISTING (6 sequences)

SEQ ID NO: 1 - DNA, 20 bp, Homo sapiens
  misc_feature: fluorescein introduced at the 5′
aggctgctccccgcgtggcc

SEQ ID NO: 2 - DNA, 20 bp, Homo sapiens
ggccacgcggggagcagcct

SEQ ID NO: 3 - DNA, 20 bp, Homo sapiens
aaaaaaaaaaaaaaaaaaaa

SEQ ID NO: 4 - DNA, 10 bp, Homo sapiens
cacgcgggga

SEQ ID NO: 5 - DNA, 15 bp, Homo sapiens
ccacgcggggagcag

SEQ ID NO: 6 - DNA, 70 bp, Homo sapiens
ccggtgtaggagctgctggtgcaggggccacgcggggagcagcctctggcattctgggagcttcatctgg

(19) United States
(12) Patent Application Publication
(10) Pub. No.: US 2006/0211135 A1
(43) Pub. Date: Sep. 21, 2006

(54) Method and apparatus for separating molecules using micro-channel

(75) Inventors: Kenichi Yamashita, Tosu-shi (JP); Hideaki Maeda, Tosu-shi (JP); Hajime Shimizu, Tosu-shi (JP); Masaya Miyazaki, Tosu-shi (JP); Hiroyuki Nakamura, Tosu-shi (JP); Yoshiko Yamaguchi, Tosu-shi (JP)

Correspondence Address: WENDEROTH, LIND & PONACK, L.L.P., 2033 K STREET N. W., SUITE 800, WASHINGTON, DC, 20006-1021, US

(73) Assignee: NATIONAL INSTITUTE OF ADVANCE INDUSTRIAL SCIENCE A, Tokyo (JP)

(21) Appl. No.: 10/545,604

(22) PCT Filed: Feb. 18, 2004
(86) PCT No.: PCT/JP04/01814

§ 371 Date: Aug. 16, 2005

(30) Foreign Application Priority Data
Feb. 18, 2003 (JP) 2003-039870

(51) Int. Cl. G01N 33/558

(52) U.S. Cl. 436/514

(57) ABSTRACT

A method for simply and efficiently separating substances by utilizing a specific flow behavior in a non-turbulent flow, i.e. a laminar flow, in a micro-channel is disclosed. A mixed solution containing at least two kinds of solute molecules which are different from each other in molecular weight and/or molecular sharp, or at least two kinds of solutions containing their respective solute molecules are flowed into a micro-channel to form a non-turbulent flow. A physical action is given to each molecule by changing the state of flow, thereby causing different behaviors among the different solute molecules. By utilizing this behavior difference, molecules of a specific kind are gathered in a specific region in the channel for separation.

### TECHNOLOGICAL FIELD

The present invention relates to a novel method for separating molecules or agglomerates of molecules such as cells by molecular species from a mixture of two kinds or more of molecules or, more particularly, to a novel method for separating different molecules or agglomerates of molecules by utilizing differences in the behaviors between two kinds or more of the solute molecules in a solution which are brought about by effecting changes in a non-turbulent flowing condition caused in a micro flow channel as well as an apparatus for carrying out the same.

### BACKGROUND TECHNOLOGY

In the preparation of a chemical substance, it is indispensable, in most cases, to undertake separation and purification of the product as the final step. In continuously conducting a reaction for the preparation of a desired chemical substance, it is also a necessary treatment to separate and purify the intermediate product as the intermediate step in order to accomplish a higher reaction rate and higher efficiency.

A great variety of methods are known heretofore as the means for such separation and purification including solvent-extraction methods using a solvent, fractionating precipitation methods from a solution, filtration methods through a filtering material, dialysis methods through a permeable membrane, fractionating distillation methods utilizing the difference of the boiling points, zone-melting methods suitable for the purification of single crystals, electrophoresis methods, chromatographic methods and so on and they are utilized as adequately selected depending on the object of separation thereof.

However, no methods for carrying out separation or purification between molecular species by utilizing laminar flows as formed in a micro flow channel are known heretofore.

### DISCLOSURE OF THE INVENTION

The present invention has been completed with an object to provide a method for separation of substances with easiness and efficiency by utilizing the characteristic action of the flowing behaviors under a non-turbulent condition, or namely, a laminar-flow condition within a micro flow channel as well as an apparatus suitable for carrying out the same.

The inventors have continued extensive investigations with regard to the relationship between a non-turbulent flowing condition within a micro flow channel and substance molecules therein and, as a result, have arrived at a discovery that, changes in the non-turbulent flowing condition accompany addition of the characteristic action on the solute molecules existing in a solution under a non-turbulent flowing condition in which the action depends on a mass of molecule or, namely, a molecular weight or a molecular configuration so that, by utilizing the same, it is possible to separate and purify, with easiness, two kinds or more of molecules having different molecular weights or molecular configurations leading to completion of the present invention on the base of this discovery.

Namely, the present invention relates to a method for separation of molecules characterized by comprising the steps of: passing a mixed solution containing at least two kinds of solute molecules having different molecular weights and/or different molecular configurations each from the other or at least two kinds of solutions each containing the respective solute molecules independently from the other through a micro flow channel under a non-turbulent flowing condition; adding a physical action to each of the molecules by modifying the flowing condition thereof thereby to cause differences in the behaviors between different kinds of the solute molecules brought about by the said action; utilizing the same to cause localization of the molecules of a specified kind only in a specified zone within the flow channel; and separating the same as well as an apparatus for separation of molecules satisfactory for carrying out the method.

The “non-turbulent flowing condition” here implied means a condition in which parallel flows in a definite direction are formed without occurrence of a turbulent flow in every portion within a cross section of the flow.

### BRIEF DESCRIPTION OF THE DRAWING

FIG. 1 is a plan view showing tracks obtained in Example 1.

FIG. 2 shows cross sectional views of the flow channel in the front and in the rear of the bent portions in Example 2.

FIG. 3 is a plan view of the micro flow channel used in Example 3.

FIG. 4 is an explanatory drawing of the main part of the property detecting sensor used in Examples 3 and 4.

FIG. 5 is a bar chart showing the results of Example 3.

FIG. 6 is a plan view of the micro flow channel used in Example 4.

FIG. 7 is a bar chart showing the results of Example 4.

### BEST MODE FOR CARRYING OUT THE INVENTION

The micro flow channel used in the method of the present invention can be constituted of a capillary tube made of an inactive material or can be provided in the form of a groove on a base plate made of an inactive material. The inactive material implied here is a material, which exhibits no reactivity with the solvents, solutes and compounds produced by the reaction, as exemplified, for example, by glass, quartz or silica, Si/SiO₂, magnesia, zirconia, alumina, apatite, silicon nitride and ceramic materials including oxides, carbides, nitrides, borides, silicides and the like of metals such as titanium, aluminum, yttrium and tungsten.

In addition, any metals, plastics and the like can also be used provided that they are inactive materials. With respect to the form of the base plate, it can usually be a flat plate but, if so desired, those having an arch-wise form, spherical form, granular form and others can be used.

The micro flow channel is prepared by engraving as a groove in a size of 1 to 1000 μm or, preferably 50 to 500 μm width and depth, or formed as a capillary tube having a comparable size. It is desirable to properly select the size depending on the viscosity and flow rate of the solution taken into consideration the hydrodynamic variables such as the Reynolds number and the like, and others. The length of this micro flow channel is, though not particularly limited provided that it is selected, corresponding to the kinds and conditions of solute molecules to be separated, in the range, usually, from 100 to 1000 mm.

Such a micro flow channel can be a commercial capillary tube used as such or can be prepared by engraving, on a base plate of an inactive material, by a mechanical means using a machine tool such as a microdrill or, alternatively, by engraving with the photolithography used in the manufacture of semiconductor integrated circuits and others followed by adhesive bonding of another base plate thereto.

When a fluid such as a liquid is introduced to flow in such an extra-fine micro flow channel, the liquid flows straightly in a definite direction or, namely, in the direction of the flow channel under a non-turbulent flowing condition. Such an extra-fine flow channel has characteristics of a short diffusion distance of solute molecules, relatively large contacting area with the wall surface, a large gradient of the flow velocity within a cross section of the flow channel, and others.

When such a micro flow channel has a portion not straightly linear like a bent portion, even though the non-turbulent flowing condition can be maintained, the solvent molecules and solute molecules contained therein are under physical actions by the centrifugal force and force of inertia at the bent portions and centrifugal force, force of inertia or collision and rebounding at the wall surface in the bent portions depending on differences in the configuration of the micro flow channel, velocity of the flow, steric structure and molecular weight of the molecules and the like. And, these actions cause a secondary flow of the solution within the flow channel or, namely, the flow having components in the direction of the flow channel and the perpendicular direction.

In the present invention, separation of the objective substances is performed by means of the molecular screening effect obtained by utilizing one or a plurality of these actions. Among the aforementioned plurality of the physical actions including the centrifugal force, force of inertia, secondary flows and others, the type of the action and the extent of the influence caused thereby depend on the kind of the solute molecules as the objective of separation.

For example, a centrifugal force acts at the bent portion of the flow channel so as to attract the heavier molecules more toward outside. Since the strength of the force depends on the weight of the solute molecules and the curvature of the curve, separation of the objective solute molecules can be accomplished by the utilization of this physical phenomenon. Furthermore, while the solute molecules in the solution are under continuous impingement of the solvent molecules and the frequency of such impingements depends on the conformation of the solute molecules, the conformation of the solute molecules is also an important factor in conducting separation in addition to the molecular weight and curvature of the bent portion so that separation can be effected on the base of the conformation of the solute molecules.

When a solution flows along a portion of the flow channel not straightly linear as in the bent portions, furthermore, a secondary flow is generated within the flow channel by means of the centrifugal force and the force of inertia acting on the solvent molecules as generated there. Accordingly, still higher performance of separation can be accomplished by jointly utilizing this phenomenon and the aforementioned force acting on the solute molecules.

In this way, localization of specified solute molecules can be accomplished in specified regions within the flow channel and such a state of localization can be maintained insofar as a non-turbulent flow condition is realized within the micro flow channel so that a solute as desired can be selectively taken out by means of a control of the flow channel structure, i.e. by forming the outlet structure of the flow channel to be branched from the aforementioned portion of localization.

In this way, according to the method of the present invention, localization of giant molecules such as proteins and the like can be efficiently accomplished based on the molecular weight and steric structure thereof by means of the molecular screening effect utilizing a micro flow channel thereby to carry out function analyses of proteins conveniently and rapidly at low cost.

According to the method of the present invention, the object can be achieved within a remarkably short time with easiness as compared with gel electrophoresis conventionally used for the same object. And, an additional advantage is obtained that objective substances such as proteins and the like alone can be selectively taken out. Furthermore, it is possible to continuously introduce the solution so that handling of a large number of samples is practicable.

According to the method of the present invention, It is also possible to utilize as an analytical means such as quantitative determination and the like by measuring the amounts of the solute molecules localized in a part within the flow channel.

When, in the method of the present invention, a mixed solution containing two kinds or more of different molecules is introduced or two kinds or more of solutions each containing different molecules are individually introduced, as keeping contact each with the other, into the micro flow channel, the mixed solution forms two kinds or more of flows having respectively different molecular concentrations or those solutions flow in a condition with formation of an interface without being intermixed each with the other. In the latter case, a complex is formed between the solute molecules of those solutions on this interface provided that they have specific affinity therebetween and, in the cases of DNAs where the base sequences are complementary or a protein and a substrate with a specific interaction therebetween, for example, changes are caused in the molecular weights or in the molecular configurations. Thereby, separation can be conducted by causing selective localization of the thus formed complex alone or analyses are conducted by utilizing the same.

In the method of the present invention, the solution can be introduced to the micro flow channel, for example, by carrying out manually with an injector while it is advantageous to undertake a mechanical means such as a syringe pump and the like to automatically carry out under control of the liquid feed rate, liquid feed pressure and others.

In the method of the present invention, separation of the objective molecules can be conducted by virtue of the simple procedure merely to pass a solution through a micro flow channel to accomplish separation within a greatly shortened time as compared with the prior art separation methods by utilizing the molecular screening effect and, furthermore, it is an analytical method of wide applicability that a great variety of separations can be accomplished by modifying the passing conditions and, in addition, there can be obtained an advantage enabling a high-performance separation such as multiple-stage separation and others by way of the design of the flow channel and high-precision separation by way of temperature control.

In the following, the best mode for carrying out the present invention is described by way of examples although the present invention is never limited by these examples in any way.

### EXAMPLE 1

The experiments were undertaken for accomplishing movement of solute molecules in a solution, when a solution is passing over a bent portion of a micro flow channel, toward the outward direction of the bent portion of the flow channel by the centrifugal force or other physical actions.

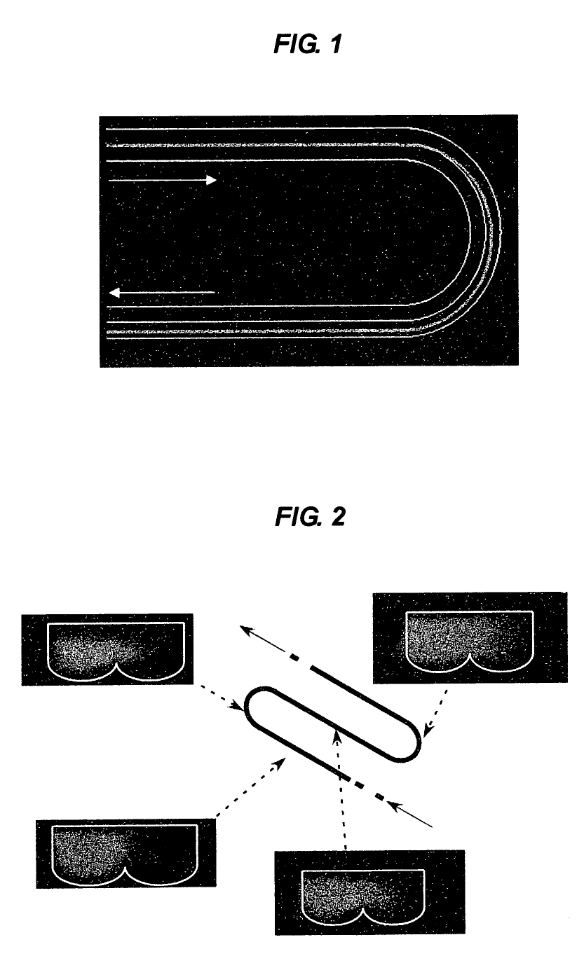

FIG. 1 is a plan view of the track drawn by the duplex DNA molecules having 20 pairs of bases with a molecular weight of 12000 found in the center portions when an aqueous solution was passed through a micro flow channel having a width of 360 μm and a depth of 200 μm in the U-shaped cross section at a velocity of 10 mm/second. The bent portion of the flow channel here had a radius of curvature of 1 mm.

It is understood from this figure that the DNA molecules formerly found on the centerline of the flow channel had moved toward the outward direction of the flow channel curve during turning of the curve by a physical action such as a centrifugal force and others. Since the value of this physical action depends on the molecular weight, molecular configuration and others, it is possible to freely modify the extent of the molecular screening effect by controlling the liquid-flowing conditions.

### EXAMPLE 2

Experiments were undertaken for observation of the condition of interface deformation when a solution was flowing in the bent portion of a micro flow channel by directly taking a picture of a cross section of the flow channel by means of a confocal laser scanning microscope.

FIG. 2 is a cross sectional view of a flow channel in the front of and in the rear of bent portions under flowing of an aqueous solution containing fluorescein as a fluorescent dye and pure water free from the same by keeping contact therebetween through an S-shaped micro flow channel having a width of 360 μm and a depth of 200 μm at a velocity of 10 mm/second.

As is understood from this figure, the interface of the two solutions moved in the outward direction by the movement of the inner side solution under the physical action received at the bent portions. This means that a secondary flow is generated within the flow channel by the outward movement of the solvent also under a physical action such as a centrifugal force and others. Accordingly, separation of substances can be performed in an increased efficiency by the combination of the molecular screening effect on the solute molecules alone and this action.

### EXAMPLE 3

Experiments were undertaken for separation and analysis by utilizing the molecular screening effect caused by the weight increase due to the formation of a complex between two solutions flowing in a micro flow channel as compared with the weight before complex formation. Detection was performed here for duplex DNAs formed sequence-selectively.

As the probe DNA, was prepared a DNA fragment having fluorecein of the fluorescent substance introduced to the 5′ terminal as follows: F-(5′)-AGGCTGCTCCCCGCGTGGCC-(3′) (wherein F is fluorecein).

As the sample DNAs were prepared two kinds of DNA fragments as follows:

(5′)-GGCCACGCGGGGAGCAGCCT-(3′) (referred to as the sample 1 hereinafter). (5′)-AAAAAAAAAAAAAAAAAAAA-(3′) (referred to as the sample 2 hereinafter).

Four solutions as a total were prepared including a solution containing no DNA fragments (referred to hereinafter as the blank solution) along with the three kinds of the DNA fragment solutions. The solutions had a solution composition of 1 pmol/μl of DNA, 5 mM phosphate buffer solution (pH 7.0) and 50 mM of sodium chloride. Three combinations including those of the probe DNA solution and the sample 1 solution, the probe DNA solution and the sample 2 solution and the probe DNA solution and the blank solution were each introduced to the micro flow channel system with a configuration as shown in FIG. 1. A liquid-introducing rate was set to be 20 μl/min.

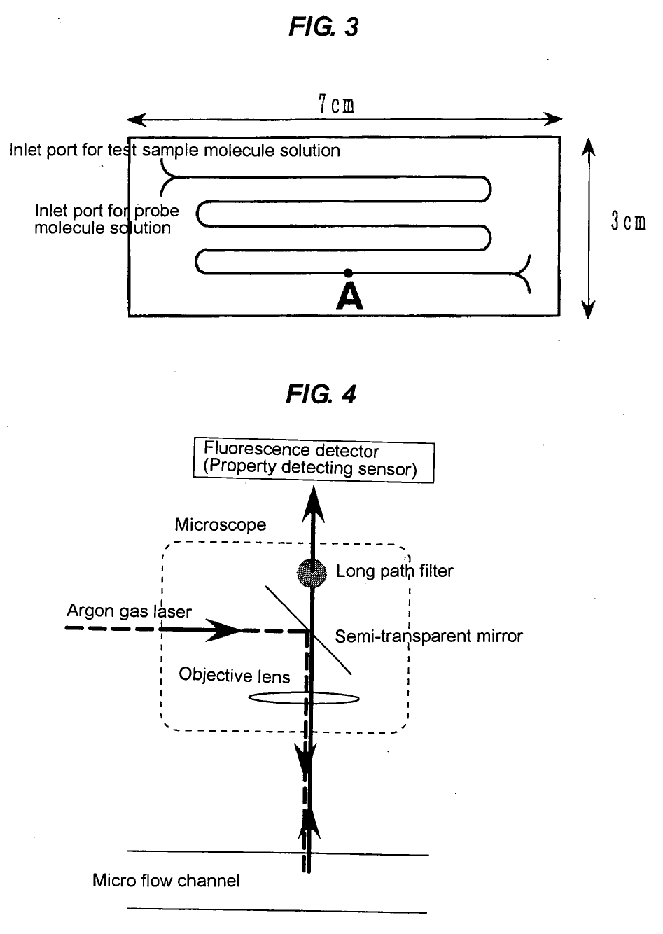

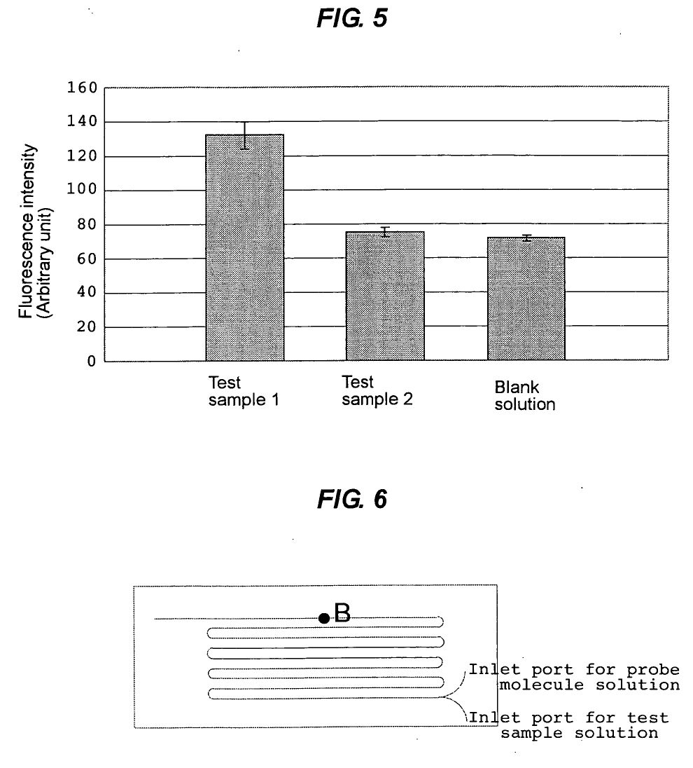

FIG. 3 shows a plan view of a micro flow channel having four bent portions. This flow channel has the same cross section as that in Example 2. FIG. 4 is an explanatory drawing showing the microscope portion including the fluorescence detector or, namely, the property detecting sensor. And, irradiation was made with a beam of an argon gas laser of 488 nm to the test sample flow channel side at the position A in the micro flow channel to cause emission of fluorescence and comparison was made for the intensities thereof which were detected by the microscope. The results are shown in FIG. 5 as a bar chart. These values are the average values obtained by ten-times measurements of the fluorescent intensities (in an arbitrary unit) and the ranges of the standard deviations are indicated with error bars. It is understood from this figure that a particularly high fluorescence response can be obtained only in the case of the sample 1 having a base sequence complementary with the probe DNA fragments as comparison was made with the other two controls.

It is understood from these results that those having specific interactions or, namely base-sequence complementary ones are converted into a heavy complex by forming a duplex chain at the interface and moved toward the test sample flow channel side by the molecular screening effect at the bent portions.

The results of the measurements have a variation coefficient of around 3% to indicate analyzability with very high reproducibility.

### EXAMPLE 4

Since, in the method of the present invention, the efficiency of separation depends on the molecular weight or the molecular size, it is possible to construct an instrument for the determination of the length of the sample DNAs from the fluorescence intensity by applying this fact to the means of Example 3. In this Example, the experiments are indicated.

The probe DNA used here was the same one as used in Example 3 and the sample DNAs prepared here included Sample 1 in Example 3 and those listed below.

(5′)-CACGCGGGGA-(3′) (referred to as the sample 3 hereinafter).

(5′)-CCACGCGGGGAGCAG-(3′) (referred to as the sample 4 hereinafter).

(5 ′)-CCGGTGTAGGAGCTGCTGGTGCAGGGGCCACGCGGGGAGCAGCCTCTG GCATTCTGGGAGCTTCATCTGG-(3′) (referred to as the sample 5 hereinafter).

Solutions were prepared each having a composition of 1 pmol/μl of DNA, 5 mM phosphate buffer solution (pH 7.0) and 5 mM of sodium chloride by using these five kinds of the DNA fragments. Four combinations including those of the probe DNA solution and the sample 1 solution, the probe DNA solution and the sample 3 solution, the probe DNA solution and the sample 4 solution and the probe DNA solution and the sample 5 solution were each introduced to the micro flow channel system with a configuration as shown in FIG. 6. In this case, a liquid-introducing rate was set to be 40 μl/minute and the temperature was 35° C.

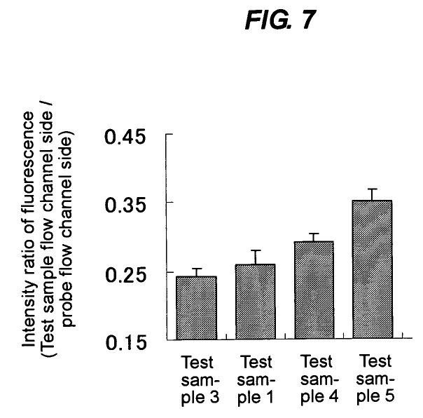

FIG. 6 is a plan view of the micro flow channel bent eight-fold as used in this Example. This flow channel had a cross sectional profile which was the same as in Example 3. Irradiation with a 488 nm argon gas laser beam was conducted at each of the test sample flowing channel side and the probe flowing channel side on the spot B in the micro flow channel to cause fluorescence emission and to measure the intensity thereof by means of a fluorescence detector making evaluation by way of the intensity ratio of the two fluorescences. The results are shown in FIG. 7 as a bar chart. These values are average values obtained by ten-times measurements of the fluorescence intensities and the ranges of the standard deviations are indicated with error bars. As is understood from this figure, the response obtained corresponded to the length of the sample DNA fragments taken as the objective of detection. Thus, it is now possible by using this device to know the size of an unknown sample DNA fragment from the ratio of the fluorescence intensities.

### INDUSTRIAL UTILIZABILITY

The present invention is applicable to the maneuvers for separation of chemical substances in general or, particularly, satisfactory for separation of high molecular-weight substances such as, for example, macromolecular substances, DNAs, proteins and the like.

1. A method for separation of molecules characterized by comprising the steps of:
passing a mixed solution containing at least two kinds of solute molecules having different molecular weights and/or different molecular configurations each from the other or at least two kinds of solutions each containing the respective solute molecules independently from the other through a micro flow channel under a non-turbulent flowing condition;
adding a physical action to the respective molecules by modifying the flowing condition thereof thereby to cause differences in the behaviors between different kinds of the solute molecules brought about by the said action; and
utilizing the same to cause localization of the molecules of a specified kind only in a specified zone within the flow channel for separation.

2. The method for separation of molecules described in claim 1 in which the physical action is selected from a secondary flow, a centrifugal force and a force of inertia.

3. The method for separation of molecules described in claim 1 in which the difference in the molecular weights or in the molecular configurations between different kinds of solute molecules is amplified by forming a complex of the molecules to be separated along the interfacial surface between the respective solutions flowing under the non-turbulent flowing condition.

4. An apparatus for separation of molecules constituted of:
a substrate plate provided with a micro flow channel engraved therein having one or at least two bent portions, of which one end serves for a sample inlet port and the other end serves for a sample outlet port; and
a sensor means for detection of physical properties disposed in correspondence to the flow at the inward side or at the outward side of the bent portions.
